(19) United States
(12) Patent Application Publication
(10) Pub. No.: US 2017/0112881 A1
(43) Pub. Date: Apr. 27, 2017

(54) Method of Treating Endometriosis

(71) Applicant: Bradley C. Robinson, North Salt Lake, UT (US)

(72) Inventor: Bradley C. Robinson, North Salt Lake, UT (US)

(73) Assignee: Predictive Therapeutics, LLC, North Salt Lake, UT (US)

(21) Appl. No.: 15/399,532

(22) Filed: Jan. 5, 2017

Related U.S. Application Data
(60) Provisional application No. 62/275,413, filed on Jan. 6, 2016.

(51) Int. Cl. A61K 35/51; A61K 9/08; A61K 9/00

(52) U.S. Cl. CPC A61K 35/51; A61K 9/0019; A61K 9/08

(57) ABSTRACT

The present invention is a method of treating or preventing endometriosis by means of administering stem cells to a patient. The stem cells are preferably drawn from umbilical cord tissue and/or umbilical cord blood and are preferably combined with a liquid carrier to form a stem cell solution. In the case of a female patent asymptomatic of endometriosis but at increased risk of developing endometriosis due to at least one endometriosis associated genetic marker being known to exist in the patient, the stem cell solution is preferably injected in the general abdominal/rectal/vaginal area of the patient. In the case of a female patent known to have endometriosis, the stem cell solution may be injected directly into endometrial tissue, and more especially into an endometrial lesion of the patient. The stem cell solution may also be used for treating other disorders such as infertility.

### CROSS REFERENCE TO RELATED APPLICATIONS

This U.S. nonprovisional utility patent application claims the benefit under 35 USC §119(e) of U.S. provisional application No. 62/275,413 filed Jan. 6, 2016 which is expressly incorporated herein in its entirety by this reference.

### FIELD OF THE INVENTION

The present invention relates to methods for use in treating those known to have or be at increased risk of endometriosis.

### BACKGROUND OF THE INVENTION

Endometriosis is a common gynecological disorder. Many therapeutics including progestins and uses of such therapeutics in the treatment of endometriosis are well known. An example of such therapeutics and methods is taught in US patent application 20080306034 to Ward and U.S. Pat. No. 8,932,993 to Ward et al. both of which are incorporated herein in their entirety by this reference. Additional examples of such therapeutics and methods is taught in U.S. Pat. Nos. 9,149,499 and 9,161,941 to Robinson both of which are incorporated herein in their entirety by this reference. Further, the use of stem cells in promoting tissue growth including reconstructive tissue growth is known in the art (see Appx A).

### SUMMARY OF THE INVENTION

The present invention is a method of treating or preventing endometriosis by means of administering stem cells to a patient. The stem cells are preferably drawn from umbilical cord tissue and/or umbilical cord blood. The stem cells are preferably provided in the form of an injectable solution or slurry such as by being mixed with a liquid carrier component (the stem cell solution). In the case of a female patent asymptomatic of endometriosis but at increased risk of developing endometriosis due to at least one endometriosis associated genetic marker being known to exist in the patient, the stem cell solution is preferably injected in the general abdominal/rectal/vaginal area of the patient. In the case of a female patent known to have endometriosis, the stem cell solution may be injected directly into the endometrial tissue, and more especially into an endometrial lesion of the patient. The stem cell solution may also be used for treating other disorders such as infertility.

### DETAILED DESCRIPTION OF THE INVENTION

Reference throughout this specification to “one embodiment” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Furthermore, the described features, structures, or characteristics of the invention may be combined in any suitable manner in one or more embodiments. In the following description, numerous specific details are included to provide a thorough understanding of embodiments of the invention. One skilled in the relevant art will recognize, however, that the invention can be practiced without one or more of the specific details, or with other methods, components, materials, and so forth. In other instances, well-known structures, materials, or operations are not shown or described in detail to avoid obscuring aspects of the invention.

A first embodiment of the invention is a method of treating an endometriosis asymptomatic patient by administering to the patient a stem cell solution. The stem cell solution is preferably injected in the general abdominal/rectal/vaginal area of the patient. Alternatively the stem cell solution may be administered generally by an IV drip application of by a combination thereof. The stem cells of the stem cell solution are preferably drawn from umbilical cord tissue and/or umbilical cord blood. The stem cell solution administration may be preceded by analyzing a DNA sample of the patient and identifying in the DNA sample at least one endometriosis associated genetic marker such as the minor allele of a SNP known to have an association with endometriosis.

A second embodiment of the invention is a method of treating a patient known to have endometriosis by administering to the patient a stem cell solution. The stem cell solution is preferably injected directly into the endometrial tissue, and more especially into an endometrial lesion of the patient. Alternatively the stem cell solution may be administered generally by an IV drip application of by a combination thereof. The stem cells of the stem cell solution are preferably drawn from umbilical cord tissue and/or umbilical cord blood. The stem cell solution administration may be preceded by analyzing a DNA sample of the patient and identifying in the DNA sample at least one endometriosis associated genetic marker such as the minor allele of a SNP known to have an association with endometriosis.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method of treating a patient comprising applying stem cells to the pelvic region of said patient.

2. The method of claim 1, wherein at least a portion of said stems are obtained from at least one of umbilical cord blood and umbilical cord tissue.

3. The method of claim 1, wherein said stem cells are provided in an injectable solution form, and wherein said injectable solution is applied by at least one of direct injection, intravenous administration, and a combination thereof.

4. The method of claim 1, wherein said stem cells are provided in an injectable solution form, and wherein said injectable solution is applied by injecting said solution into endometrial tissue of said patient.

5. The method of claim 4, wherein said endometrial tissue defines at least one endometrial lesion of said patient.

6. The method of claim 1, wherein the DNA of said patient is known to have at least one endometriosis associated genetic marker therein.

7. The method of claim 1, wherein said patient is endometriosis asymptomatic.

8. The method of claim 1, wherein said patient is infertile.

9. A method of treating endometriosis in a patient comprising injecting a stem cell solution into the endometrial tissue of said patient.

10. The method of claim 9, wherein at least a portion of said stems are obtained from at least one of umbilical cord blood and umbilical cord tissue.

11. The method of claim 9, wherein said stem cells are also administered to said patient intravenously.

12. The method of claim 9, wherein said endometrial tissue defines at least one endometrial lesion of said patient.

13. The method of claim 9, wherein the DNA of said patient is known to have at least one endometriosis associated genetic marker therein.

14. The method of claim 9, wherein said patient is at least one of endometriosis asymptomatic and infertile.

15. A method of treating a patient known to have at least one endometriosis associated genetic marker in the DNA thereof comprising applying stem cells to the pelvic region of said patient.

16. The method of claim 15, wherein at least a portion of said stems are obtained from at least one of umbilical cord blood and umbilical cord tissue.

17. The method of claim 15, wherein said stem cells are provided in an injectable solution form, and wherein said injectable solution is applied by at least one of direct injection, intravenous administration, and a combination thereof.

18. The method of claim 15, wherein said stem cells are provided in an injectable solution form, and wherein said injectable solution is applied by injecting said solution into endometrial tissue of said patient.

19. The method of claim 18, wherein said endometrial tissue defines at least one endometrial lesion of said patient.

20. The method of claim 15, wherein said patient is at least one of endometriosis asymptomatic and infertile.
